(19) United States
(12) Patent Application Publication
(10) Pub. No.: US 2008/0102473 A1
(43) Pub. Date: May 1, 2008

(54) Method and system to improve contrast in lateral flow assay

(76) Inventors: Julie E. Fouquet, Portola Valley, CA (US); Daniel B. Roitman, Menlo Park, CA (US)

Correspondence Address: Kathy Manke;Avago Technologies Limited, 4380 Ziegler Road, Fort Collins, CO, 80525, US

(21) Appl. No.: 11/591,647

(22) Filed: Oct. 31, 2006

(51) Int. Cl. G01N 33/53; C12M 3/00

(52) U.S. Cl. 435/7.1; 435/287.2

(57) ABSTRACT

A lateral flow assay includes one or more optically-reactive test regions that are examined using light propagating at a wavelength that matches or nearly matches an absorption wavelength associated with each optically-reactive test region. The presence or absence of a color or absorption in each optically-reactive test region may be determined by an individual examining each optically-reactive test region or by one or more detectors that detect fluorescence from one or more optically-reactive test regions or detect light transmitted through or reflected off one or more optically-reactive test regions.

### BACKGROUND

Lateral flow assays and other types of colorimetric assays are used for a variety of diagnostic tests, including food safety tests, water quality tests, and medical tests such as home pregnancy and diabetes tests. FIG. 1 is a graphic illustration of a lateral flow assay in accordance with the prior art. Lateral flow assay 100 includes wick region 102, detection region 104, and holding region 106. Holding region 106 is used to hold assay 100 when applying a liquid test sample to wick region 102 or examining assay 100 to determine the results.

Wick region 102 and detection region 104 are typically made of a porous material. When a liquid test sample is applied to wick region 102, wick region 102 conveys the liquid by capillary action into detection region 104, as indicated by arrow 110. Optically-reactive test region 108 absorbs light, reflects light, or produces fluorescence when one or more test substances are present or absent from the liquid test sample. For example, optically-reactive test region 108 changes color when region 108 reacts to or binds with a target molecule or microorganism in the liquid test sample. The presence or absence of a color in test region 108 is used to determine the results of a particular test. For example, a color or colored shape in optically-reactive test region 108 indicates a positive pregnancy test with many home pregnancy tests.

A person typically reads or analyzes optically-reactive test region 108 using light emitted from a broadband light source, such as a white light source. FIG. 2 is a graph of a spectrum of a broadband light source. As shown in FIG. 2, spectrum 200 includes a wide range of wavelengths. Certain wavelength(s) of light present in the broadband light source can interact with the material in optically-reactive test region 108. As a result of the presence or absence of a test substance in the test sample, test region 108 can change its optical behavior at a particular wavelength associated with the test substance. Typically, however, the optical behavior at other wavelengths is not altered by the presence of the test substance. Consequently, the contrast between the color of the material in detection region 104 and the color of test region 108 can be low. The low contrast can make it more difficult to detect a color change in test region 108.

### SUMMARY

In accordance with the invention, methods and systems to increase contrast in a lateral flow assay are provided. A lateral flow assay includes one or more optically-reactive test regions that are examined using light propagating at a wavelength that matches or nearly matches an absorption wavelength associated with each optically-reactive test region. The light may be emitted by one or more narrowband light sources or one or more broadband light sources with overlying narrowband filters to narrow the emission spectrum of each broadband light source. The presence or absence of a color or absorption in each optically-reactive test region may be determined by an individual examining each optically-reactive test region or by one or more detectors that detect fluorescence from one or more optically-reactive test regions or detect light transmitted through or reflected off one or more optically-reactive test regions. Each detector may include a narrowband filter overlying an input of the detector in order to narrow the detection spectrum of the detector.

### BRIEF DESCRIPTION OF THE DRAWINGS

FIG. 1 is a graphic illustration of a lateral flow assay in accordance with the prior art;

FIG. 2 is a graph of a spectrum of a broadband light source;

FIG. 3 is a flowchart of a first method for increasing contrast in a lateral flow assay;

FIG. 4 is a flowchart of a second method for increasing contrast in a lateral flow assay;

FIG. 5 is a graph of light emitted from a narrowband light source in an embodiment in accordance with the invention;

FIG. 6 is a block diagram of a first system for examining a lateral flow assay in an embodiment in accordance with the invention;

FIG. 7 is a graph of light transmitted through a lateral flow assay in accordance with the embodiment shown in FIG. 6;

FIG. 8 is a block diagram of a second system for examining a lateral flow assay in an embodiment in accordance with the invention;

FIG. 9 is a graph of light reflected or scattered off a lateral flow assay in accordance with the embodiment shown in FIG. 8;

FIG. 10 is a block diagram of a third system for examining a lateral flow assay in an embodiment in accordance with the invention;

FIG. 11 is a graph of light fluorescing from a lateral flow assay in accordance with the embodiment shown in FIG. 10;

FIG. 12 is a block diagram of a fourth system for examining a lateral flow assay in an embodiment in accordance with the invention;

FIG. 13 is a graph of light associated with the lateral flow assay in accordance with the embodiment shown in FIG. 12;

FIG. 14 is a block diagram of a light source in an embodiment in accordance with the invention;

FIG. 15 is a block diagram of a detector in an embodiment in accordance with the invention; and

FIG. 16 is a block diagram of a detector system in an embodiment in accordance with the invention.

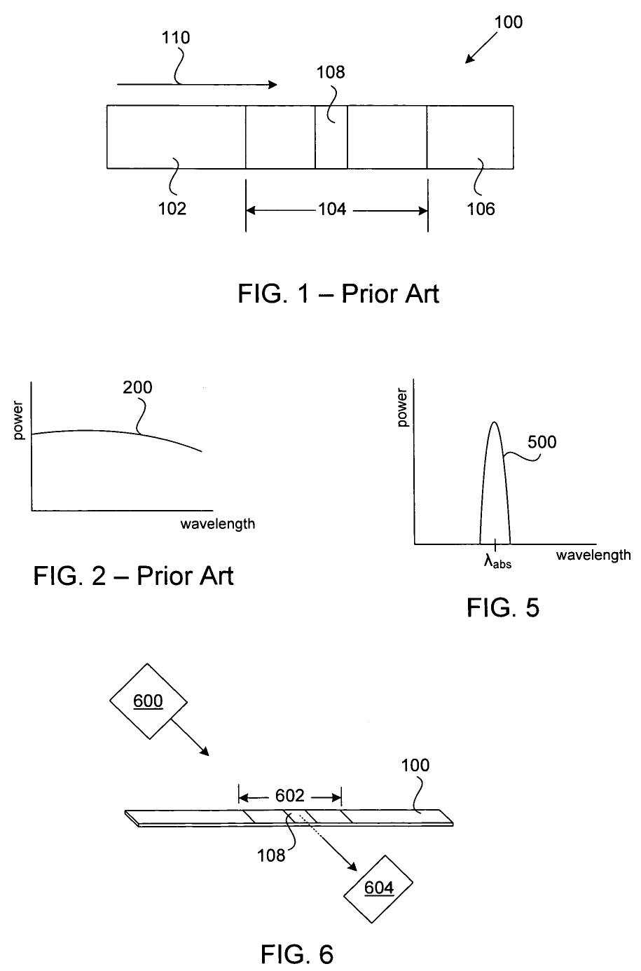

### DETAILED DESCRIPTION

The following description is presented to enable embodiments of the invention to be made and used, and is provided in the context of a patent application and its requirements. Various modifications to the disclosed embodiments will be readily apparent, and the generic principles herein may be applied to other embodiments. Thus, the invention is not intended to be limited to the embodiments shown but is to be accorded the widest scope consistent with the appended claims. Like reference numerals designate corresponding parts throughout the figures.

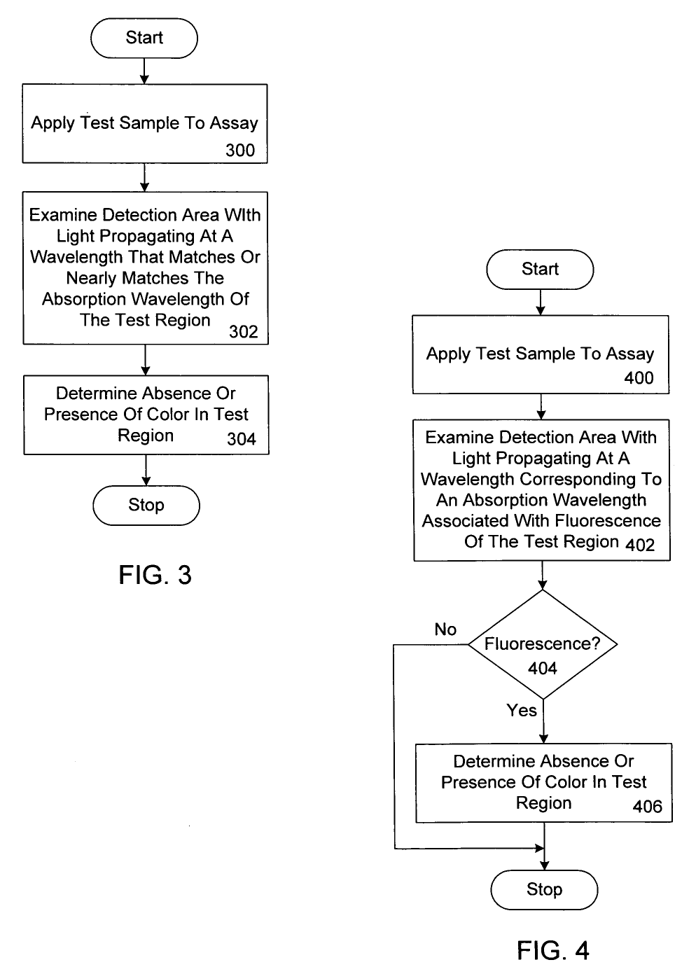

FIG. 3 is a flowchart of a first method for increasing contrast in a lateral flow assay. The embodiment shown in FIG. 3 is described in conjunction with a single test region in an assay. Other embodiments in accordance with the invention may provide two or more test regions in an assay.

Initially a liquid test sample is applied to the wick region of the assay, as shown in block 300. The liquid test sample may be applied, for example, by dipping the wick region of the assay in a liquid test sample or by placing drops of the liquid test sample onto the wick region. Once the wick region has conveyed the liquid test sample to the detection region of the assay, the detection region is examined with light propagating at a wavelength or wavelengths that match or nearly match an absorption wavelength of light associated with the optically-reactive test region (block 302).

Examining the detection region with light propagating at a wavelength that matches or nearly matches the wavelength of light that is absorbed by the optically-reactive test region increases the contrast between the optically-reactive test region and the color of the material in the detection region when the optically-reactive test region is activated by a molecule or substance in the test sample. The absorption wavelength or wavelengths associated with the optically-reactive test region correspond to one or more wavelengths associated with the peak absorption wavelength of the optically-reactive test region in an embodiment in accordance with the invention.

The presence or absence of one or more colors or colored shapes is then determined at block 304. A person examines the detection region for the presence of one or more colors at all possible wavelengths in an embodiment in accordance with the invention. In another embodiment in accordance with the invention, a detection system examines the detection region for the presence of one or more colors at all possible wavelengths.

Referring to FIG. 4, there is shown a second method for increasing contrast in a lateral flow assay. The embodiment shown in FIG. 4 is described in conjunction with a single test region in an assay. Other embodiments in accordance with the invention may provide two or more test regions in an assay.

Initially a liquid test sample is applied to the wick region of the assay, as shown in block 400. The liquid test sample may be applied, for example, by dipping the wick region of the assay in a liquid test sample or by placing drops of the liquid test sample onto the wick region. Once the wick region has conveyed the liquid test sample to the detection region of the assay, the detection region is examined with light propagating at or near a wavelength that corresponds to an absorption wavelength associated with fluorescence of the optically-reactive test region (block 402).

By way of example only, a narrowband light source having a wavelength associated with the peak absorption of the fluorescent test region may be emitted towards the detection region. The narrowband light source is implemented with a light-emitting diode (LED), a resonant-cavity LED, or a semiconductor laser in an embodiment in accordance with the invention. In another embodiment in accordance with the invention, a narrowband filter overlies an output of a broadband light source such that light source emits light corresponding to peak absorption of the optically-reactive test region.

Returning to FIG. 4, a determination is made at block 404 as to whether an emission produced by the optically-reactive test region is propagating at a fluorescence wavelength. If so, the presence or absence of a color is determined at block 406. A person or a detection system examines the detection region for light at all possible fluorescence wavelengths in an embodiment in accordance with the invention.

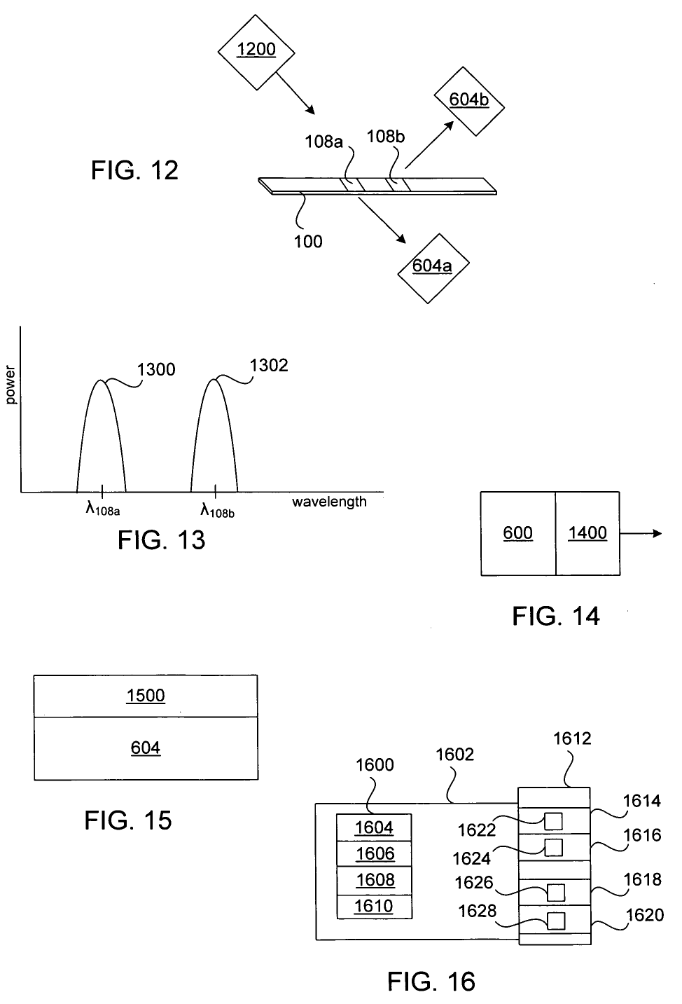

The eyes of a person are used to determine the presence or absence of color the one or more optically-reactive test regions in an embodiment in accordance with the invention. In another embodiment in accordance with the invention, the presence or absence of color in the one or more optically-reactive test regions is determined by one or more detectors. A detector detects light transmitting through, reflecting off, or fluorescing by a test region. As discussed earlier, a narrowband filter may overlie an input to a detector in order to narrow the detection spectrum of the detector. FIG. 6, FIG. 8, FIG. 10, FIG. 12, and FIGS. 14-16 describe various embodiments of detectors and detection systems that can be used to examine a lateral flow assay.

FIG. 5 is a graph of light emitted from a narrowband light source in an embodiment in accordance with the invention. Spectrum 500 is narrow compared to the spectrum of the broadband light source shown in FIG. 2. Since the absorption wavelength of an optically-reactive test region is known or predetermined, the wavelength range of spectrum 500 is tailored to match or nearly match the absorption wavelength (λ_{abs}) of the optically-reactive test region.

Referring to FIG. 6, there is shown a block diagram of a first system for examining a lateral flow assay in an embodiment in accordance with the invention. Light source 600 emits light towards assay 100 at a wavelength that corresponds to the absorption wavelength of optically-reactive test region 108. In the embodiment of FIG.6, the absorption wavelength corresponds to the peak absorption wavelength associated with optically-reactive test region 108. One or more lenses or mirrors (not shown) may be used to direct light towards test region 108.

Light source 600 is implemented as a narrowband light source, such as a light-emitting diode or a semiconductor laser, in an embodiment in accordance with the invention. In another embodiment in accordance with the invention, light source 600 is implemented as a broadband light source that includes a narrowband filter overlying the output of the light source. The broadband light source and narrowband filter are described in more detail in conjunction with FIG. 14.

Detection region 602 in assay 100 is formed with a transparent material so that light not absorbed by test region 108 is transmitted through test region 108. Detector 604 is positioned to detect the light transmitted through optically-reactive test region 108 and to detect the presence or absence of a color or absorption in an embodiment in accordance with the invention. In another embodiment in accordance with the invention, detector 604 is not used and an individual examines optically-reactive test region 108 with his or her eyes to detect the presence or absence of a color or absorption in test region 108.

Detector 604 is implemented as a complementary metal oxide semiconductor device in an embodiment in accordance with the invention. A narrowband filter may overlie an input of detector 604 in order to narrow the detection spectrum of detector 604. A detector and narrowband filter are described in more detail in conjunction with FIG. 15.

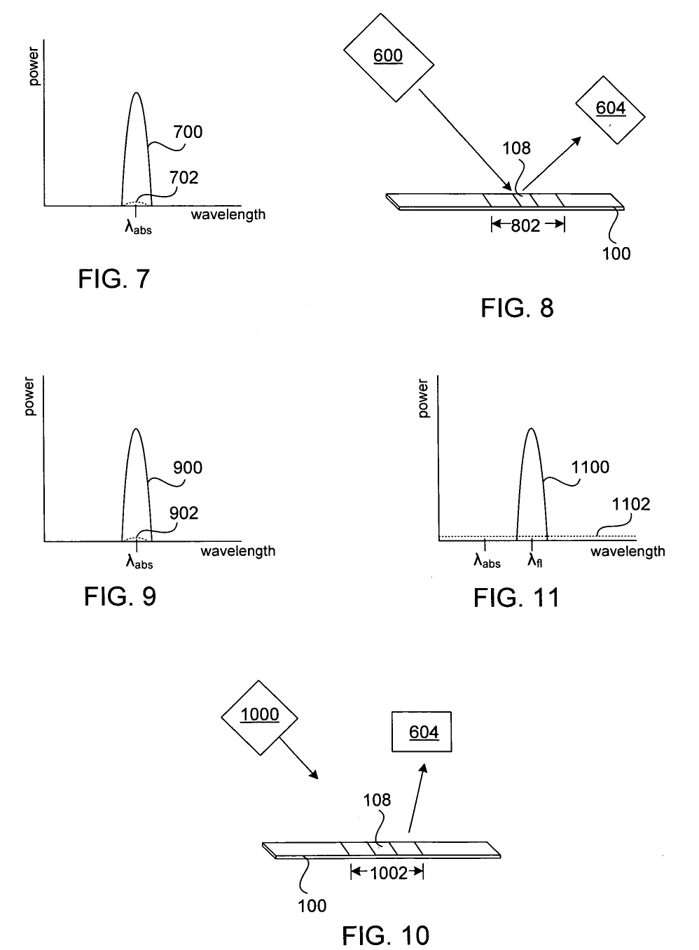

FIG. 7 is a graph of light transmitted through a lateral flow assay in accordance with the embodiment shown in FIG. 6. When the target molecule, substance, or microorganism is not present in the liquid test sample, optically-reactive test region 108 in the detection region 602 of assay 100 does not absorb any light emitted from light source 600 and all or nearly all of the light is transmitted through detection region 602. Spectrum 700 in FIG. 7 represents the amount of light transmitted through the detection region when the test sample does not include the target molecule, substance, or microorganism. Spectrum 700 peaks at the absorption wavelength (λ_{abs}), an emission wavelength chosen for light source 600 based on the known absorption wavelength associated with optically-reactive test region 108. By way of example only, wavelength (λ_{abs}) corresponds to the peak absorption wavelength associated with optically-reactive test region 108 in the embodiment of FIG. 6.

When the target molecule, substance, or microorganism is present in the liquid test sample, optically-reactive test region 108 in detection region 602 absorbs all or nearly all of the light emitted by light source 600. The absence of the light is indicated by dashed line 702 in FIG. 7. Selecting an emission wavelength and spectrum for light source 600 that equals or nearly equals wavelength (λ_{abs}) increases the fraction of incident light absorbed by test region 108, thereby making it easier for detector 604 to detect the presence or absence of light.

Referring to FIG. 8, there is shown a block diagram of a second system for examining a lateral flow assay in an embodiment in accordance with the invention. Detection region 802 in assay 100 is formed with a reflective or opaque material such that light not absorbed by optically-reactive test region 108 reflects or scatters off optically-reactive test region 108. Detector 604 is positioned to detect the reflected or scattered light and determine the presence or absence of a color or absorption in test region 108 in an embodiment in accordance with the invention. In another embodiment in accordance with the invention, detector 604 is not used and an individual examines test region 108 using his or her eyes to detect the presence or absence of color or absorption in optically-reactive test region 108.

FIG. 9 is a graph of light reflected or scattered off a lateral flow assay in accordance with the embodiment shown in FIG. 8. When the target molecule, substance, or microorganism is not present in the liquid test sample, optically-reactive test region 108 does not absorb any light and all or nearly all of the light reflects or scatters off test region 108. Spectrum 900 in FIG. 9 represents the amount of reflected or scattered light when the test sample does not include the target molecule or microorganism. Spectrum 900 peaks at the absorption wavelength (λ_{abs}), an emission wavelength chosen for light source 600 based on the known absorption wavelength associated with optically-reactive test region 108. By way of example only, wavelength (λ_{abs}) corresponds to the peak absorption wavelength associated with optically-reactive test region 108 in the embodiment of FIG. 8.

When the target molecule, substance, or microorganism is present in the liquid test sample, optically-reactive test region 108 in detection region 802 absorbs all or nearly all of the light emitted by light source 600. The absence of the light is indicated by dashed line 902 in FIG. 9. Selecting an emission wavelength and spectrum for light source 600 that equals or nearly equals wavelength (λ_{abs}) increases the fraction of the incident light absorbed by test region 108, thereby making it easier for detector 604 to detect the presence or absence of light.

Referring to FIG. 10, there is shown a block diagram of a third system for examining a lateral flow assay in an embodiment in accordance with the invention. Optically-reactive test region 108 in detection region 1002 of assay 100 includes a reagent that fluoresces when the target molecule, substance, or microorganism is present in the liquid test sample in an embodiment in accordance with the invention. Light source 1000 emits light towards assay 100 at a wavelength that corresponds to an absorption wavelength (λ_{abs}) associated with the fluorescence of test region 108. Detector 604 is positioned to detect the light fluorescing from test region 108 and determine the presence or absence of a color in an embodiment in accordance with the invention.

Light source 1000 is implemented as a narrowband light source, such as a light-emitting diode or a semiconductor laser, in an embodiment in accordance with the invention. In another embodiment in accordance with the invention, light source 1000 is implemented as a broadband light source that includes a narrowband filter overlying the output of the light source. The broadband light source and narrowband filter are described in more detail in conjunction with FIG. 14.

FIG. 11 is a graph of light fluorescing from a lateral flow assay in accordance with the embodiment shown in FIG. 10. Optically-reactive test region 108 fluoresces when the target molecule, substance, or microorganism is present in the liquid test sample. When the target molecule, substance, or microorganism is not present in the liquid test sample, test region 108 does not fluoresce. Spectrum 1100 represents the amount of fluorescence light present when the test sample includes the target molecule or microorganism. Spectrum 1100 peaks at wavelength (λ_{fl}), which corresponds to the peak fluorescence wavelength associated with optically-reactive test region 108.

When the target molecule, substance, or microorganism is present in the liquid test sample, optically-reactive test region 108 reacts by absorbing all or nearly all of the light emitted by light source 1000. The absence of the light is indicated by dashed line 1102 in FIG. 11, where dashed line 1102 represents a broadband noise floor across the spectral range of FIG. 11. Selecting an emission wavelength and spectrum for light source 1100 that equals or nearly equals wavelength (λ_{abs}) reduces interference with the fluorescence light produced by test region 108, thereby making it easier for detector 604 to detect the presence or absence of fluorescence light.

Referring to FIG. 12, there is shown a block diagram of a fourth system for examining a lateral flow assay in an embodiment in accordance with the invention. Light source 1200 emits light towards optically-reactive test regions 108a, 108b. The light propagates at wavelengths associated with the absorption wavelengths for both test regions 108a, 108b.

Light source 1200 is implemented as one or more narrowband light sources in an embodiment in accordance with the invention. In another embodiment in accordance with the invention, light source 1200 is implemented as one or more broadband light sources that include a narrowband filter overlying an output of each light source. And in yet another embodiment in accordance with the invention, light source 1200 is implemented as a broadband light source with an overlying filter that passes light propagating at two or more wavelengths.

The absorption wavelength associated with test region 108a is different from the absorption wavelength of test region 108b in an embodiment in accordance with the invention. Optically-reactive test region 108a absorbs light when the target molecule, substance, or microorganism is present in the liquid test sample. When the target molecule or microorganism is not present in the sample, the light emitted by light source 1200 is transmitted through test region 108a. Detector 604a is used to detect the presence or absence of absorption in test region 108a.

Optically-reactive test region 108b also absorbs light when a second target molecule, substance, or microorganism is present in the liquid test sample in an embodiment in accordance with the invention. When the second target molecule, substance, or microorganism is not present in the sample, the light emitted by light source 1200 reflects off test region 108b. Detector 604b is used to detect the presence or absence of absorption in test region 108b. Detectors 604a, 604b each include one or more filters (not shown) to narrow the spectra detected by detectors 604a, 604b in an embodiment in accordance with the invention.

Other embodiments in accordance with the invention may be implemented differently than the embodiment shown in FIG. 12. For example, optically-reactive test regions 108a, 108b may react similarly when the target molecules, substances, or microorganisms are present in the test sample. Both test regions may produce fluorescence light or reflect light. Moreover, more than two optically-reactive test regions may be used in an assay. And finally, the two or more test regions may have the same or different absorption wavelengths.

FIG. 13 is a graph of light associated with the lateral flow assay in accordance with the embodiment shown in FIG. 12. Spectrum 1300 represents the light reflected off, transmitted through, or fluorescence from optically-reactive test region 108a while spectrum 1302 represents the light reflected off, transmitted through, or fluorescence from optically-reactive test region 108b. The wavelength ranges for spectra 1300, 1302 do not overlap in an embodiment in accordance with the invention. The wavelength ranges may overlap partially or completely in other embodiments in accordance with the invention.

As discussed in conjunction with FIGS. 6, 8, 10, and 12, the light source may be implemented as one or more narrowband light sources, such as light-emitting diodes or semiconductor lasers, or as one or more broadband light sources that each include a narrowband filter overlying the output of the broadband light source. FIG. 14 is a block diagram of a light source in an embodiment in accordance with the invention. Narrowband filter 1400 overlies the output of light source 600 and is used to narrow the emission spectrum of light source 600. Light source 600 is implemented as a broadband light source in an embodiment in accordance with the invention.

Narrowband filter 1400 may be a single peak or multi-peak filter, depending on the number of optically-reactive test regions and associated absorption wavelengths. Narrowband filter 1400 is implemented as a dielectric stack filter in an embodiment in accordance with the invention. Dielectric stack filters are designed to have particular spectral properties, including the number of peak wavelengths and the particular wavelengths that are peak wavelengths. For example, in the embodiment shown in FIG. 13, narrowband filter 1400 is designed to have one peak at the absorption wavelength (λ₁₀₈ₐ) associated with test region 108a and another peak at the absorption wavelength (λ_{108b}) for test region 108b.

FIG. 15 is a block diagram of a detector in an embodiment in accordance with the invention. Narrowband filter 1500 overlies an input of detector 604 in order to narrow the detection spectrum of detector 604. Embodiments in accordance with the invention can use one or more detectors with a different filter overlying the input of each detector.

Narrowband filter 1500 is implemented a single peak or multi-peak filter, depending on the number of optically-reactive test regions and associated absorption wavelengths. Narrowband filter 1500 may be used, for example, when a broadband light source is used to examine one or more optically-reactive test regions in an assay in order to narrow the detection spectrum of detector 604.

Referring to FIG. 16, there is shown a block diagram of a detector system in an embodiment in accordance with the invention. Detector array 1600 is housed or placed in container 1602. Detector array 1600 includes four detectors 1604, 1606, 1608, 1610 in an embodiment in accordance with the invention. Detector array 1600 can include any given number of detectors in other embodiments in accordance with the invention.

Metal mask or baffle 1612 is placed over the opening of container 1602. Baffle 1612 includes openings 1614, 1616, 1618, 1620 that include filters 1622, 1624, 1626, 1628, respectively. The number of filters equals the number of detectors in detector array 1600 in an embodiment in accordance with the invention.

Filters 1622, 1624, 1626, 1628 allow light propagating at a specific wavelength or a wavelength range to enter container 1602 in an embodiment in accordance with the invention. Filters 1622, 1624, 1626, 1628 in openings 1614, 1616, 1618, 1620, respectively, allow detector array 1600 to detect light propagating at the specific wavelength or wavelength range when a broadband light source is used to examine one or more optically-reactive test regions or when the environment surrounding detector array 1600 has conditions that can reduce the ability of detector array 1600 to properly detect the absence or presence of color or absorption in one or more optically-reactive test regions.

1. A method for increasing a contrast between a material in a detection region of a lateral flow assay and an optically-reactive test region in the detection region, the method comprising:
emitting light towards the optically-reactive test region, wherein the light propagates at a wavelength that matches or nearly matches an absorption wavelength associated with the optically-reactive test region;
determining the absence or presence of a color in the optically-reactive test region.

2. The method of claim 1, wherein determining the absence or presence of a color in the optically-reactive test region comprises determining the absence or presence of a color in the optically-reactive test region using light reflecting off the optically-reactive test region.

3. The method of claim 1, wherein determining the absence or presence of a color in the optically-reactive test region comprises determining the absence or presence of a color in the optically-reactive test region using light transmitting through the optically-reactive test region.

4. The method of claim 1, wherein determining the absence or presence of a color in the optically-reactive test region comprises determining the absence or presence of fluorescence light emitted from the optically-reactive test region.

5. The method of claim 1, wherein the absorption wavelength of the optically-reactive test region comprises a wavelength that matches or nearly matches a peak absorption wavelength associated with the optically-reactive test region.

6. The method of claim 1, further comprising narrowing a wavelength spectrum of the light emitted towards the optically-reactive test region.

7. A system, comprising:
a lateral flow assay comprising an optically-reactive test region; and
a light source operable to propagate light at a narrow range of wavelengths including a wavelength that matches or nearly matches an absorption wavelength associated with the optically-reactive test region.

8. The system of claim 7, wherein the light source comprises a narrowband light source operable to propagate light at a narrow range of wavelengths including a wavelength that matches or nearly matches an absorption wavelength associated with the optically-reactive test region.

9. The system of claim 7, wherein the light source comprises a broadband light source with a narrowband filter overlying an output of the broadband light source in order to narrow an emission spectrum of the broadband light source such that light propagates at a narrow range of wavelengths including a wavelength that matches or nearly matches an absorption wavelength associated with the optically-reactive test region.

10. The system of claim 7, wherein the absorption wavelength comprises a wavelength associated with a peak absorption wavelength for the optically-reactive test region.

11. The system of claim 7, further comprising a detector operable to receive light from the optically-reactive test region.

12. The system of claim 11, wherein the detector detects fluorescence light emitted by the optically-reactive test region.

13. The system of claim 11, further comprising a narrowband filter configured to narrow a wavelength spectrum input into the detector.

14. The system of claim 13, wherein light reflecting from the optically-reactive test region and propagating at or near the absorption wavelength is transmitted through the narrowband filter.

15. The system of claim 13, wherein light transmitting through the optically-reactive test region and propagating at or near the absorption wavelength is transmitted through the narrowband filter.

16. A system for examining a lateral flow assay comprising an optically-reactive test region, the system comprising:
a light source operable to propagate light towards the lateral flow assay at a narrow range of wavelengths including a wavelength that matches or nearly matches an absorption wavelength associated with the optically-reactive test region; and
a detector operable to receive light from the lateral flow assay.

17. The system of claim 16, wherein the light source comprises a narrowband light source operable to propagate light towards the lateral flow assay at a narrow range of wavelengths including a wavelength that matches or nearly matches the absorption wavelength associated with the optically-reactive test region.

18. The system of claim 17, wherein the narrowband light source comprises one of a light-emitting diode, a resonant-cavity light-emitting diode, and a semiconductor laser.

19. The system of claim 16, wherein the light source comprises a broadband light source and a narrowband filter overlying an output of the broadband light source to narrow an emission spectrum of the broadband light source such that light propagates towards the lateral flow assay at a narrow range of wavelengths including a wavelength that matches or nearly matches an absorption wavelength associated with the optically-reactive test region.

20. The system of claim 16, wherein the absorption wavelength comprises a wavelength that matches or nearly matches a peak absorption wavelength of the optically-reactive test region.

21. The system of claim 16, wherein the detector further comprises a narrowband filter overlying an input of the detector.

22. The system of claim 21, wherein light reflecting from the optically-reactive test region and propagating at or near the absorption wavelength is transmitted through the narrowband filter.

23. The system of claim 21, wherein light transmitting through the optically-reactive test region and propagating at or near the absorption wavelength is transmitted through the narrowband filter.

24. The system of claim 16, further comprising a container surrounding the detector, wherein the container includes a removable baffle comprising surfaces to block the transmission of unwanted light and one or more openings at one end of the container.
